(19) United States
(12) Patent Application Publication
(10) Pub. No.: US 2026/0217756 A1
(43) Pub. Date: Jul. 30, 2026

(54) Peptide synthesis method involving sterically hindered tri-tert-butyl-tryptophan (Tbt) residue

(71) Applicant: Amicoat AS, Tromsø (NO)

(72) Inventors: Jacobus Johannes Eksteen, Tromso (NO); John Sigurd Svendsen, Tromso (NO); Florence Malmedy, Brussels (BE); Jonathan Guevara, Brussels (BE)

(21) Appl. No.: 19/145,240

(22) PCT Filed: Jan. 5, 2024
(86) PCT No.: PCT/EP2024/050221

§ 371 Date: Jul. 2, 2025

(30) Foreign Application Priority Data
Jan. 5, 2023 (GB) 2300173.8

(51) Int. Cl. C07K 1/02

(52) U.S. Cl. CPC C07K 1/023

(57) ABSTRACT

The invention is directed to a method of peptide synthesis comprising reacting a compound of Formula (I) or a salt thereof with a carbodiimide reagent to form an O-acylisourea intermediate; reacting the O-acylisourea intermediate with an additive to form an activated ester; and reacting the activated ester with an amino-containing moiety which is an amino acid, a peptide or a salt thereof comprising an amino group, wherein the amino group forms an amide bond with the carbonyl marked * in Formula (I); wherein the compound of Formula (I) has the structure: and wherein R¹ and R² are as defined in the disclosure. The invention is also directed to a compound of Formula (III) as defined in the disclosure or a salt thereof.

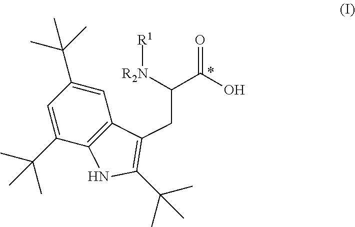

### TECHNICAL FIELD

The invention is directed to a method of peptide synthesis and a method of making a target peptide.

### BACKGROUND

At laboratory scale, many coupling strategies are available for the production of peptides. However, most of these are too expensive for commercial scale production. Coupling reactions between amino acids are, almost exclusively, facilitated by activation of the carboxylic acid of the incoming amino acid. For example, 0-(1H-Benzotriazol-1-yl)-N,N,N′,N′-tetramethyluronium hexafluorophosphate (HTBU) is an efficient activator that gives minimal racemization. However, the cost of HTBU is high.

It would be desirable to be able to use lower cost activators without compromising on yield or reaction rate. However, lower cost activators are not suitable for all peptide coupling strategies. In particular, depending on the candidate amino acid to be activated, some activators may not allow coupling at acceptable rates or with acceptable yield. Slow reactions in particular may be associated with undesirable racemization/epimerisation.

Badlands et al. Tetrahedron Letters 58 (2017) 4391-4394 discloses a series of reactions with amide-bond forming reagents, including the carbodiimides N,N′-diisopropylcarbodiimide (DIC) and 1-ethyl-3-(3-dimethylaminopropyl)carbodiimide (EDC) with the additives HOPO and Oxyma. As illustrated in Badlands et al., couplings involving sterically hindered carboxylic acids can be problematic. In particular, Badlands et al. reported that the sterically hindered carboxylic acid 2,6-dimethylbenzoic acid required forcing conditions of a prolonged reaction time at elevated temperature (70° C. for 48 h) to provide a modest yield of the desired amide product when using DIC/HOPO, and that all of the other coupling reagents tested provided inadequate yields for this sterically hindered carboxylic acid (Table 3, Entries 13 and 14). Badlands et al. also reported that there was negligible conversion of 2,6-dimethylbenzoic acid to the amides using DIC/HOPO at lower temperatures of 20° C. (page 4392, penultimate sentence).

### SUMMARY OF THE INVENTION

The present inventors have surprisingly found that a carbodiimide/additive approach can be used to couple an amino-containing moiety as defined herein to a compound of Formula (I) in high yields favourable for a commercial process and at relatively low temperatures, without observing significant epimerisation, despite the extremely sterically bulky side chain of the tri-tert-butyl-tryptophan (Tbt) residue in Formula (I).

In one aspect, the invention provides a method of peptide synthesis. The method may be a step in the synthesis of a target peptide. The method of peptide synthesis of this aspect comprises:
reacting a compound of Formula (I) or a salt thereof with a carbodiimide reagent to form an O-acylisourea intermediate;
reacting the O-acylisourea intermediate with an additive to form an activated ester; and
reacting the activated ester with an amino-containing moiety which is an amino acid, a peptide or a salt thereof comprising an amino group, wherein the amino group forms an amide bond with the carbonyl marked * in Formula (I);
wherein the compound of Formula (I) has the structure:

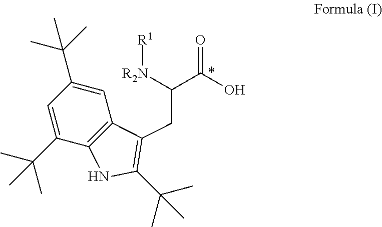

wherein R₁ is a protecting group, a peptide or an amino acid; and
wherein R₂ is H, an alkylsilyl group or a protecting group.

Another aspect provides a method of amino acid coupling. In the context of this aspect, “amino acid coupling” means that one amino acid or peptide is coupled to another amino acid or peptide. That is, each of the coupling partners may independently be an amino acid or a peptide. Embodiments of other aspects of the invention described herein apply, mutatis mutandis, to this aspect of the invention. The method of amino acid coupling of this aspect comprises:
reacting a compound of Formula (I) or a salt thereof with a carbodiimide reagent to form an O-acylisourea intermediate;
reacting the O-acylisourea intermediate with an additive to form an activated ester; and
reacting the activated ester with an amino-containing moiety which is an amino acid, a peptide or a salt thereof comprising an amino group, wherein the amino group forms an amide bond with the carbonyl marked * in Formula (I);
wherein the compound of Formula (I) has the structure:

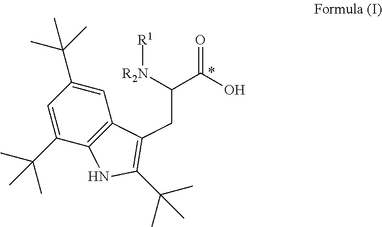

wherein R₁ is a protecting group, a peptide or an amino acid; and
wherein R₂ is H, an alkylsilyl group or a protecting group.

In a third aspect, the invention provides a method for making a target peptide comprising:
reacting a compound of Formula (I) or a salt thereof with a carbodiimide reagent to form an O-acylisourea intermediate;
reacting the O-acylisourea intermediate with an additive to form an activated ester; and
reacting the activated ester with an amino-containing moiety which is an amino acid, a peptide or a salt thereof comprising an amino group, wherein the amino group forms an amide bond with the carbonyl marked * in Formula (I);
wherein the compound of Formula (I) has the structure:

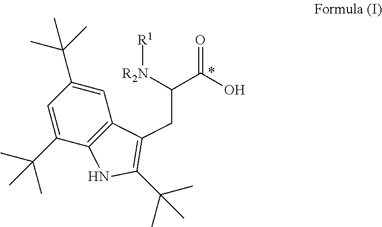

wherein R₁ is a protecting group, a peptide or an amino acid; and
wherein R₂ is H, an alkylsilyl group or a protecting group.

Embodiments of other aspects of the invention described herein apply, mutatis mutandis, to the third aspect of the invention. The reactions recited above may form the target peptide or a precursor to the target peptide. For example, subsequent steps to remove one or more protecting groups may be necessary to provide the target peptide.

### DETAILED DESCRIPTION

### Compound of Formula (I)

Formula (I) comprises an extremely sterically bulky tri-tert-butyl-tryptophan (Tbt) residue and has the structure:

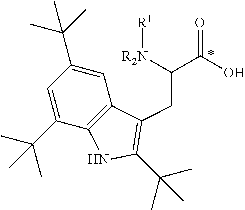

The inventors have unexpectedly found that, in spite of the extremely high steric bulk of the Tbt side chain, amino acids or peptides can be coupled to the compound of Formula (I) in high yields and at low temperature using a carbodiimide/additive approach.

In Formula (I), R₁ is: a protecting group (typically an amine protecting group), a peptide or an amino acid. Optionally, the peptide or the amino acid may themselves comprise one or more protecting groups, such as on their N-terminal amino groups.

As used herein the term peptide includes peptidomimetics, although true peptides are preferred. A peptidomimetic is typically characterised by retaining the polarity, three dimensional size and functionality (bioactivity) of its peptide equivalent but wherein the peptide bonds have been replaced, often by more stable linkages. By ‘stable’ is meant more resistant to enzymatic degradation by hydrolytic enzymes. Generally, the bond which replaces the amide bond (amide bond surrogate) conserves many of the properties of the amide bond, e.g. conformation, steric bulk, electrostatic character, possibility for hydrogen bonding etc. Chapter 14 of “Drug Design and Development”, Krogsgaard, Larsen, Liljefors and Madsen (Eds) 1996, Horwood Acad. Pub provides a general discussion of techniques for the design and synthesis of peptidomimetics. In the present case, where the target peptide reacts with a membrane rather than the specific active site of an enzyme, some of the problems described of exactly mimicing affinity and efficacy or substrate function are not relevant and a peptidomimetic can be readily prepared based on a given peptide structure or a motif of required functional groups. Suitable amide bond surrogates include the following groups: N-alkylation (Schmidt, R. et al., Int. J. Peptide Protein Res., 1995, 46, 47), retro-inverse amide (Chorev, M and Goodman, M., Acc. Chem. Res, 1993, 26, 266), thioamide (Sherman D. B. and Spatola, A. F. J. Am. Chem. Soc., 1990, 112, 433), thioester, phosphonate, ketomethylene (Hoffman, R. V. and Kim, H. O. J. Org. Chem., 1995, 60, 5107), hydroxymethylene, fluorovinyl (Allmendinger, T. et al., Tetrahydron Lett., 1990, 31, 7297), vinyl, methyleneamino (Sasaki, Y and Abe, J. Chem. Pharm. Bull. 1997 45, 13), methylenethio (Spatola, A. F., Methods Neurosci, 1993, 13, 19), alkane (Lavielle, S. et. al., Int. J. Peptide Protein Res., 1993, 42, 270) and sulfonamido (Luisi, G. et al. Tetrahedron Lett. 1993, 34, 2391).

The term ‘amino acid’ may thus conveniently be used herein to refer to the equivalent sub-units of a peptidomimetic compound. Moreover, peptidomimetics may have groups equivalent to the R groups of amino acids.

As is discussed in the text book referenced above, as well as replacement of amide bonds, peptidomimetics may involve the replacement of larger structural moieties with di- or tripeptidomimetic structures and in this case, mimetic moieties involving the peptide bond, such as azole-derived mimetics may be used as dipeptide replacements. Peptidomimetics and thus peptidomimetic backbones wherein the amide bonds have been replaced as discussed above are, however, preferred.

Suitable peptidomimetics include reduced peptides where the amide bond has been reduced to a methylene amine by treatment with a reducing agent e.g. borane or a hydride reagent such as lithium aluminium-hydride. Such a reduction has the added advantage of increasing the overall cationicity of the molecule.

Other peptidomimetics include peptoids formed, for example, by the stepwise synthesis of amide-functionalised polyglycines. Some peptidomimetic backbones will be readily available from their peptide precursors, such as peptides which have been permethylated, suitable methods are described by Ostresh, J. M. et al. in Proc. Natl. Acad. Sci. USA(1994) 91, 11138-11142. Strongly basic conditions will favour N-methylation over O-methylation and result in methylation of some or all of the nitrogen atoms in the peptide bonds and the N-terminal nitrogen. Preferred peptidomimetic backbones include polyesters, polyamines and derivatives thereof as well as substituted alkanes and alkenes. The peptidomimetics will preferably have N and C terminii which may be modified as discussed herein.

Preferably, the term “amino acid” as used herein refers to proteinogenic (genetically encoded) amino acids. Preferably, the term peptide in R₁ and the amino-containing moiety refers to a peptide formed from proteinogenic amino acids.

R₁ typically comprises 1 to 10 amino acids, preferably 1 to 5 or 1 to 3 amino acids, most preferably 1 amino acid.

A wide choice of protecting groups suitable for amino acids are known (see, for example, Greene, T. W. and Wuts, P. G. M., Protective Groups in Organic Synthesis, 3rd ed., Wiley: New York, 1999 and Isidro-Llobet et al., Chem. Rev. 2009, 109, 6, 2455-2504).

Suitable amine protecting groups (which may also be referred to as amino protecting groups) include carbobenzoxy (also known as benzyloxycarbonyl and designated Z or Cbz), t-butoxycarbonyl (also designated Boc), 4-methoxy-2,3,6-trimethylbenzene sulphonyl (Mtr), 9-fluorenylmethoxy-carbonyl (also designated Fmoc), 2,2,2-trichloroethoxycarbonyl (Troc), 2,4-dimethoxybenzyl (Dmb), 2-hydroxy-4-methoxybenzyl (Hmb) and 2-Fmoc-oxy-4-methoxybenzyl (FmocHmb). These protecting groups may themselves be R₁ or one or more of these protecting groups may be present on R₁ when R₁ is a peptide or an amino acid.

Suitable carboxyl protecting groups which may, for example be employed include readily cleaved ester groups such as benzyl (Bn), p-nitrobenzyl (pNb), pentachlorophenyl (PClP), pentafluorophenyl (Pfp) or t-butyl (tBu) groups as well as the coupling groups on solid supports, for example methyl groups linked to polystyrene. Other suitable carboxyl protecting groups include 4-{N-[1-(4,4-dimethyl-2,6-dioxocyclohexylidene)-3-methylbutyl]amino}benzyl ester (Dmab), allyloxycarbonyl (Alloc) and 2-phenylisopropyl (2-PhiPr).

Thiol protecting groups include p-methoxybenzyl (Mob), trityl (Trt), acetamidomethyl (Acm), tert-butyl (tBu), tert-butylthio (tButhio) and monomethoxytrityl (Mmt) groups.

Amine protecting groups such as Boc and carboxyl protecting groups such as tBu may be removed simultaneously by acid treatment, for example with trifluoroacetic acid. Thiol protecting groups such as Trt may be removed selectively using an oxidation agent such as iodine.

Preferably, R₁ is a peptide or an amino acid, optionally comprising one or more protecting groups, such as on its N-terminal amino group. The amino acid may be a cationic amino acid AA₁.

Preferably, R₁ is a cationic amino acid AA₁ optionally comprising one or more protecting groups, such as on its N-terminal amino group.

AA₁ is preferably, lysine or arginine but may be histidine or any non genetically coded or modified amino acid carrying a positive charge at pH 7.0.

Suitable non-genetically coded amino acids and modified amino acids which can provide a cationic amino acid include analogues of lysine, arginine and histidine such as homolysine, ornithine, diaminobutyric acid, diaminopimelic acid, diaminopropionic acid and homoarginine as well as trimethylysine and trimethylornithine, 4-aminopiperidine-4-carboxylic acid, 4-amino-1-carbamimidoylpiperidine-4-carboxylic acid and 4-guanidinophenylalanine.

Most preferably, R₁ is arginine optionally comprising one or more protecting groups, such as on its N-terminal amino group.

In Formula (I), R₂ is selected from H, an alkylsilyl group, or a protecting group (typically an amine protecting group). The alkylsilyl group may be a mono(C₁-C₆ alkyl)silyl, di(C₁-C₆ alkyl)silyl or tri(C₁-C₆ alkyl)silyl group. Preferably, the alkylsilyl group is a tri(C₁-C₆ alkyl)silyl group, more preferably a tri(C₁-C₃ alkyl)silyl group. Each alkyl group may be the same or different, preferably the same. If present as R₂, the alkylsilyl group is preferably trimethyl silyl. The protecting group may be an amine protecting group as defined above. For example, the protecting group may be Cbz, Boc, Mtr, Troc, Dmb, Hmb or FmocHmb.

R₂ is preferably H. That is, the compound of Formula (I) preferably has the structure

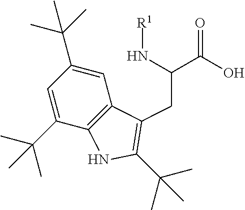

In the processes of the invention, the compound of Formula (I) may preferably be provided in the form of a salt, preferably in the form of an acid addition salt, more preferably a HCl salt. Other suitable acid addition salts are defined below in relation to the amino-containing moiety.

### Amino-Containing Moiety

The amino-containing moiety is an amino acid, a peptide or a salt thereof. The amino acid or peptide of the amino-containing moiety may optionally contain one or more protecting groups and/or a C-terminal capping group. The amino acid or peptide of the amino-containing moiety may optionally be silylated. Suitable silylating agents are disclosed in WO 2009/065836. For example, suitable silylating agents are N-trialkylsilyl amines or N-trialkylsilyl amides, such as those selected from the group consisting of: N,O-bis(trimethylsilyl)acetamide, N,O-bis(trimethylsilyl)trifluoroacetamide, hexamethyldisilazane, N-methyl-N-trimethylsilylacetamide (MSA), N-methyl-N-trimethylsilyltrifluoroacetamide, N-(trimethylsilyl)acetamide, N-(trimethylsilyl)diethylamine, N-(trimethylsilyl)dimethylamine, 1-(trimethylsilyl)imidazole and 3-(trimethylsilyl)-2-oxazolidone. Silylation may improve solubility of the amino-containing moiety, for example in polar organic solvents such as polar aprotic solvents, e.g. dimethylacetamide. During silylation one or more functional groups in the amino-containing moiety having an active hydrogen, such as amino, hydroxyl, mercapto or carboxyl groups, react with the silylating agent. The silylated amino-containing moiety then comprises one or more silyl groups (such as trialkylsilyl, typically tri(C₁-C₃)alkyl groups such as trimethylsilyl) bonded to said functional groups.

The amino-containing moiety may preferably be provided in the form of a salt, such as an acid addition salt. Compounds having at least one basic centre, for example in the side-chain of an amino acid of the amino-containing moiety, can form acid addition salts. Suitable acid addition salts may be formed with strong inorganic acids, such as mineral acids, for example sulfuric acid, phosphoric acid or a hydrohalic acid; with organic carboxylic acids; or with organic sulfonic acids, such as (C₁-C₄) alkyl or arylsulfonic acids which are unsubstituted or substituted, for example by halogen, for example methyl- or p-toluene-sulfonic acid. Examples of organic carboxylic acids include: alkanecarboxylic acids of 1 to 4 carbon atoms (for example acetic acid) which are unsubstituted or substituted, for example, by halogen such as chloroacetic acid; saturated or unsaturated dicarboxylic acids, for example oxalic, malonic, succinic, maleic, fumaric, phthalic or terephthalic acid; hydroxycarboxylic acids, for example ascorbic, glycolic, lactic, malic, tartaric or citric acid; and benzoic acid.

Preferred, salts of the amino-containing moiety include hydrochloride, trifluoroacetate or acetate salts, most preferably hydrochloride salts. For example, when the amino containing moiety is a compound of Formula (VI), the amino containing moiety may preferably be in the form of a salt wherein the side chain of AA₃ is protonated.

The amino-containing moiety typically comprises a reactive amino group, such as an alpha-amino group.

The amino-containing moiety typically comprises 1 to 10 amino acids, preferably 1 to 5 or 1 to 3 amino acids, most preferably 1 amino acid.

As set out above, suitable protecting groups for amino acids are well known and the protecting groups listed above may also be used in the amino-containing moiety.

Suitable C-terminal capping groups are of formula —X—Y—Z, wherein the left hyphen denotes the point of attachment to the carbon of the C-terminal carbonyl and X, Y and Z are defined as for Formula (VI) below. In other words, if present, capping group —X—Y—Z is attached to the remainder of the amino-containing moiety as follows:

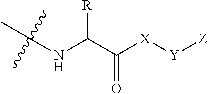

wherein R denotes the side chain of the C-terminal amino acid. Preferably, —X—Y—Z together is the group —NHCH₂CH₂Ph.

Preferably, the amino-containing moiety is a compound of Formula (VI) or a salt thereof:

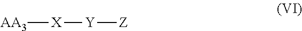

wherein AA₃ is a cationic amino acid, preferably lysine or arginine but may be histidine or any non genetically coded or modified amino acid carrying a positive charge at pH 7.0;
X is a N atom, which may be, but preferably is not, substituted by a branched or unbranched C₁-C₁₀ alkyl or aryl group (such as a C₄-C₁₀ aryl group), e.g. methyl, ethyl or phenyl, and this alkyl or aryl group may incorporate up to 2 heteroatoms selected from N, O and S;
Y represents a group selected from —Rₐ—R_{b}—, —Rₐ—R_{b}—R_{b}— and —R_{b}—R_{b}—Rₐ wherein
Rₐ is C, O, S or N, preferably C, and
R_{b} is C; each of Rₐ and R_{b} may be substituted by C₁-C₄ alkyl groups or unsubstituted, preferably Y is —Rₐ—R_{b}— (in which Rₐ is preferably C) and preferably this group is not substituted, when Y is —Rₐ—R_{b}—R_{c}— or R_{b}—R_{b}—Rₐ then preferably one or more of Rₐ and R_{b} is substituted; and
Z is a group comprising 1 to 3 cyclic groups each of 5 or 6 non-hydrogen atoms (preferably C atoms), 2 or more of the cyclic groups may be fused; one or more of the rings may be substituted and these substitutions may, but will typically not, include polar groups, suitable substituting groups include halogens, preferably bromine or fluorine and C₁-C₄ alkyl groups; the Z moiety incorporates a maximum of 15 non-hydrogen atoms, preferably 5-12, most preferably it is phenyl;
the bond between Y and Z is a covalent bond between Rₐ or R_{b} of Y and a non-hydrogen atom of one of the cyclic groups of Z.

The compound of Formula (VI) may optionally contain one or more protecting groups and/or be silylated. The discussion of silylation and suitable silylating agents above applies equally when the amino-containing moiety is a compound of Formula (VI). The protecting groups listed above may also be used when the amino-containing moiety is a compound of Formula (VI).

Suitable non-genetically coded amino acids and modified amino acids which can provide a cationic amino acid include analogues of lysine, arginine and histidine such as homolysine, ornithine, diaminobutyric acid, diaminopimelic acid, diaminopropionic acid and homoarginine as well as trimethylysine and trimethylornithine, 4-aminopiperidine-4-carboxylic acid, 4-amino-1-carbamimidoylpiperidine-4-carboxylic acid and 4-guanidinophenylalanine.

Preferably, Y is —Rₐ—R_{b}— as defined above, more preferably wherein Rₐ and R_{b} are unsubstituted, most preferably wherein Rₐ and R_{b} are both carbon atoms. In other words, Y is most preferably —CH₂CH₂—.

Preferably, —X—Y—Z together is the group —NHCH₂CH₂Ph.

Most preferably, AA₃ is arginine.

In other preferred cases, the amino-containing moiety is an amino acid comprising AA₃ or a peptide comprising AA₃ as the N-terminal amino acid, or a salt thereof, optionally wherein the amino acid or peptide comprise one or more protecting groups and/or a C-terminal capping group. The definitions of AA₃ above in the context of Formula (VI) apply equally in this case. The C-terminal capping group may have the structure —X—Y—Z, and the preferred definitions of each of —X—Y—Z set out above also apply equally to this case. The amino acid comprising AA₃ or the peptide comprising AA₃ as the N-terminal amino acid may optionally be silylated, and the discussion of silylation and suitable silylating agents above applies equally.

The compounds of the invention and those used and made in/by the methods of the invention (e.g. of Formula (I), the target peptide and the amino-containing moiety) may include all enantiomeric forms, both D and L amino acids and enantiomers resulting from chiral centers within the amino acid R groups and moieties Y or Z.

More preferably, the amino-containing moiety is arginine or a salt thereof, optionally comprising one or more protecting groups and/or a C-terminal capping group. In this case, the C-terminal capping group may be of formula —X—Y—Z, and is preferably —NHCH₂CH₂Ph.

### Carbodiimide Reagent and Additives

Suitable carbodiimide reagents and additives for peptide coupling are disclosed in Ayman EI-Faham and Fernando Alberto, Chem. Rev. 2011, 111, 6557-6602. The carbodiimide reagents and additives disclosed in Tables 1 and 2 of this document are suitable for use in the processes described herein.

The carbodiimide reagent may be a compound of Formula (II) or a salt thereof:

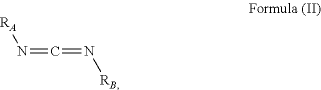

wherein R_{A} and R_{B} are each independently selected from an organic group containing 1-30 non-hydrogen atoms.

Preferably, R_{A} and R_{B} are each independently selected from the group consisting of:
C₁-C₁₀ alkyl (preferably C₁-C₆ alkyl, more preferably C₁-C₄ alkyl) optionally substituted with a mono- or di-(C₁-C₁₀ alkyl)amino group, preferably wherein the optional substituent is a mono- or di-(C₁-C₆ alkyl)amino group, more preferably wherein the optional substituent is (CH₃)₂N—;
C₃-C₈ cycloalkyl, preferably C₅-C₆ cycloalkyl;
aryl, preferably 6- to 10-membered aryl; more preferably phenyl; (aryl)C₁-C₁₀alkyl, preferably (6- to 10-membered aryl)C₁-C₆ alkyl, more preferably benzyl; or
(C₃-C₈ heterocycloalkyl)C₁-C₁₀alkyl wherein the heterocycloalkyl group is optionally substituted with one or more C₁-C₁₀ alkyl groups, preferably (C₃-C₆ heterocycloalkyl)C₁-C₄alkyl wherein the heterocycloalkyl group is optionally substituted with one or more C₁-C₄ alkyl groups, more preferably 2-morpholinoethyl or (2,2-dimethyl-1,3-dioxolan-4-yl)methyl.

The term “alkyl”, as used herein alone or as part of another group such as (heterocycloalkyl)alkyl or (aryl)alkyl, is intended to include both branched and straight-chain saturated aliphatic hydrocarbon groups having the specified number of carbon atoms. For example, “C₁-C₁₀ alkyl” is intended to include C₁, C₂, C₃, C₄, C₅, C₆, C₇, C₈, C₉, and C₁₀ alkyl groups. Preferred alkyl group are C₁-C₆ alkyl groups, more preferably C₁-C₄ alkyl groups. Examples of suitable alkyl groups include methyl (Me), ethyl (Et), propyl (e.g., n-propyl and isopropyl), butyl (e.g., n-butyl, isobutyl, t-butyl), and pentyl (e.g., n-pentyl, isopentyl, neopentyl).

The term “alkynyl” or is intended to include hydrocarbon chains of either straight or branched configuration having one or more, preferably one to three, more preferably one, carbon-carbon triple bonds that may occur in any stable point along the chain. For example, “C₂-C₆ alkynyl” is intended to include C₂, C₃, C₄, C₅, and C₆ alkynyl groups; such as ethynyl, propynyl, butynyl, pentynyl, and hexynyl.

The term “cycloalkyl” refers to cyclized alkyl groups, including mono-, bi- or poly-cyclic ring systems. “C₃-C₈ cycloalkyl” is intended to include C₃, C₄, C₅, C₆, C₇, and C cycloalkyl groups, including monocyclic, bicyclic, and polycyclic rings.

Examples of suitable cycloalkyl groups include cyclopropyl, cyclobutyl, cyclopentyl, cyclohexyl, and norbornyl. Spiro and bridged cycloalkyl groups are included in the definition of “cycloalkyl”.

The term cycloalkenyl refers to non-aromatic cyclized alkenyl groups, comprising one or more carbon-carbon double bonds. “Cycloalkenyl” includes groups such as cyclopentenyl and cyclohexenyl as well as groups with more than one double bond such as 1,3- and 1,4-cyclohexadienyl.

The term “aryl” refers to monocyclic or polycyclic (including bicyclic and tricyclic) aromatic hydrocarbons, including, for example, phenyl, naphthyl, anthracenyl, and phenanthranyl. In one embodiment, the term “aryl” denotes monocyclic and bicyclic aromatic groups containing 6 to 10 carbons in the ring portion (such as phenyl or naphthyl including 1-naphthyl and 2-naphthyl).

The terms “heteroaryl” or “heteroaromatic ring” refer to monocyclic or polycyclic (including bicyclic and tricyclic) aromatic hydrocarbons wherein one or more carbon ring members have been replaced with a heteroatom, such as O, N or S. Typically, the heteroaryl or heteroaromatic ring contains no more than 4 nitrogens, no more than 2 oxygens and no more than 2 sulfurs. The heteroaryl preferably contains 1-4 heteroatoms. The term 5-10 membered heteroaryl means there are 5-10 ring members which may be selected from carbon or a heteroatom as set out above. Preferred heteroaryl/heteroaromatic rings contain 5 or 6 ring members. Examples of suitable heteroaryl groups include pyridyl, pyrimidinyl, pyrazinyl, pyridazinyl, triazinyl, furyl, quinolyl, isoquinolyl, thienyl, imidazolyl, thiazolyl, indolyl, pyrroyl, oxazolyl, benzofuryl, benzothienyl, benzthiazolyl, isoxazolyl, pyrazolyl, triazolyl, tetrazolyl, indazolyl, 1,2,4 thiadiazolyl, isothiazolyl, purinyl, carbazolyl, benzimidazolyl, indolinyl, benzodioxolanyl, and benzodioxane.

The term “heterocycloalkyl” refers to saturated cyclized alkyl groups where one or more carbon ring members have been replaced with a heteroatom, such as 0, N or S. Typically, the heterocycloalkyl ring contains no more than 4 nitrogens, no more than 2 oxygens and no more than 2 sulfurs. “C₃ to C₈ heterocycloalkyl” is intended to include C₃, C₄, C₅, C₆, and C₇ and C₈ heterocycloalkyl groups. Examples of heterocycloalkyl groups include oxetanyl, tetrahydrofuranyl, 1,3-dioxolanyl, tetrahydropyranyl, azetidinyl, pyrrolidinyl, piperidinyl, morpholinyl, and piperazinyl.

In the moieties (C₃-C₈ heterocycloalkyl)C₁-C₁₀alkyl and (aryl)C₁-C₁₀alkyl the heterocycloalkyl or aryl groups are bonded to the C₁-C₁₀alkyl group which is in turn bonded to the remainder of Formula (II). The C₁-C₁₀alkyl group is preferably a C₁-C₆ alkyl group, more preferably a C₁-C₄ alkyl group.

Unless otherwise indicated, the aryl and heterocycloalkyl groups can be attached through any available carbon or nitrogen by replacement of a hydrogen on said carbon or nitrogen.

For example, the carbodiimide may be selected from N,N′-dicyclohexylcarbodiimide (DCC), N,N′-diisopropylcarbodiimide (DIC), 1-ethyl-3-(3-dimethylaminopropyl)carbodiimide, N-cyclohexyl-N′-isopropylcarbodiimide (CIC), N-tert-butyl-N′-methylcarbodiimide, N-tert-butyl-N′-ethylcarbodiimide, N,N′-dicyclopentylcarbodiimide, 1,3-bis(2,2-dimethyl-1,3-dioxolan-4-ylmethyl)carbodiimide, N-ethyl-N′-phenylcarbodiimide, N-phenyl-N′-isopropylcarbodiimide, N-cyclohexyl-N′-(2-morpholinoethyl)carbodiimide, N-benzyl-N′-cyclohexylcarbodiimide or a salt thereof, such as a salt selected from 1-ethyl-3-(3-dimethylaminopropyl)carbodiimide hydrochloride (EDC, which is also referred to herein as EDC.HCl), 1-[3-(dimethylamino)propyl]-3-ethylcarbodiimide methiodide (CAS number 22572-40-3) or N-Cyclohexyl-N′-(2-morpholinoethyl)carbodiimide methyl-p-toluenesulfonate (CAS number 2491-17-0).

Preferred, carbodiimide reagents include EDC.HCl and 1-ethyl-3-(3-dimethylaminopropyl)carbodiimide more preferably EDC.HCl.

Optionally, the carbodiimide reagent may be immobilised on a solid support, for example a polymeric support, such as insoluble polymeric supports for solid-phase peptide synthesis (SPPS). For example, polymer-bound EDC.HCl, polymer bound 1-(3-Dimethylaminopropyl)-3-ethylcarbodiimide or polymer bound N-benzyl-N′ cyclohexylcarbodiimide (all of which are commercially available) may be used in the processes of the invention. However, the process is preferably conducted in solution rather than involving SPPS.

The structure of the O-acylisourea intermediate is determined by the choice of carbodiimide reagent. For example, the O-acylisourea intermediate may be a compound of Formula (III) or a salt thereof, wherein R₁ and R₂ are as defined above for Formula (I) and R_{A} and R_{B} are as defined above for Formula (II)

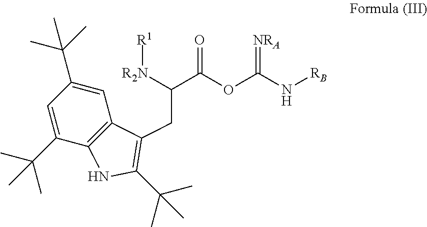

In another aspect, the invention is directed to a compound of Formula (III) as defined herein. Embodiments of other aspects of the invention described herein apply, mutatis mutandis, to this aspect of the invention.

The additive reacts with the O-acylisourea intermediate to form an activated ester. The activated ester contains a good leaving group on the carbonyl marked * in Formula (I)/Formula (V). This means that the leaving group can effectively leave the ternary intermediate which is formed which is when the amino-containing moiety reacts with the activated ester. The leaving group is provided by the additive.

The pKa of the additive (e.g. the compound of Formula IV HO-R_{c}) may be less than 8, such as from 3-7.5 or 3.5-7 or 4-6.5.

The pKa values in the present disclosure may be as determined in water at 25° C., optionally an organic co-solvent such as an equal volume mixture of methanol, dioxane and acetonitrile may be added if the compound (e.g. the additive or an acid) is sparingly soluble in water. Techniques for measuring pKa values are known, e.g. as described in Babid et al., Trends in Analytical Chemistry, Vol. 26, No. 11, 2007, pages 1043-1061. Preferably, pKa values as referred to herein are determined by potentiometric titration.

The additive may be a compound of Formula IV or a salt thereof:

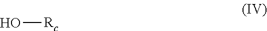

wherein R_{C} is an organic group comprising 1-30 non-hydrogen atoms. R_{C} may be a group which can stabilise a negative charge on the oxygen shown in Formula (IV), for example by delocalisation of the charge.

R_{C} may be a phenyl group, optionally substituted with one or more halogen atoms (preferably wherein the halogen atoms are each independently selected from F and Cl) or one or more electron withdrawing groups such as nitro. Preferably, the phenyl group is substituted. Examples of suitable additives having this structure are pentafluorophenol, 2,3,5-trichlorophenol and 4-nitrophenol.

Alternatively, the compound of Formula (IV) may be an N-hydroxy compound wherein R_{C} is an organic group containing 1-30 non-hydrogen atoms and at least one nitrogen atom and wherein the hydroxyl group in Formula (IV) is bonded to the R_{C} group through said nitrogen atom. For example, the N-hydroxy compound may comprise a hydroxyimino group (e.g. as in Compounds 1-19 and 28-73 of Table 1), an N-hydroxy-triazole group (e.g. as in Compounds 87-91 of Table 1), an N-hydroxy-tetrazole group (e.g. as in Compound 26 of Table 1), an N-hydroxybenzimidazole group (e.g. as in Compounds 23 and 24 of Table 1), an N-hydroxyindolin-2-one group (e.g. as in Compound 27 of Table 1), a N-hydroxypyridinone group (e.g. as in Compound 22 of Table 1), an N-hydroxypyrrolidine-2,5-dione group (e.g. as in Compounds 20-21 of Table 1), a 4-aza-, 5-aza-, 6-aza- or 7-aza-1-hydroxybenzotriazole group (e.g. as in Compounds 80 and 82-84 of Table 1), a N-hydroxybenzotriazole group (e.g. as in Compounds 77-79 and 81 of Table 1) or the group

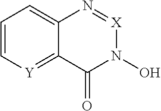

wherein Y is CH and X is N, or Y is N and X is CH, or Y is N and X is N (as in Compounds 25, 85 and 86 of Table 1).

The additive may be a compound of Formula VIII, or a salt thereof,

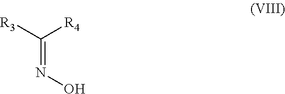

wherein R₃ is selected from the group consisting of cyano, —C(O)OR₅, 5-10 membered heteroaryl optionally substituted with one or more C₁-C₆ alkyl groups, C₂-C₆ alkynyl, nitro, aryl (preferably 6- to 10-membered aryl, more preferably phenyl), —SO₂R₆, —OSO₂R₆, —CONR₉R₁₀, —C(O)—NH—OH, and C(═N—OH)R₇;
R₄ is selected from the group consisting of —C(O)R₃, —CONR₉R₁₀, aryl (aryl (preferably 6- to 10-membered aryl, more preferably phenyl) optionally substituted with one or more halogens, 5-10 membered heteroaryl optionally substituted with one or more C₁-C₆ alkyl groups, cyano, —C(S)NR₉R₁₀, nitro, C₁-C₆ alkyl optionally substituted with aryl (preferably 6- to 10-membered aryl, more preferably phenyl), —S(O)NR₁₁R₁₂, —C(O)—NH—OH, halogen (preferably Cl), —NH₂, and hydrogen;
R₅ is C₁-C₆ alkyl;
R₆ is —NH₂, C₁-C₆ alkyl or C₁-C₆ haloalkyl;
R₇ is halogen (preferably Cl) or C₁-C₆ alkyl;
R₈ is C₁-C₆ alkoxy optionally substituted with a C₃-C₈ heterocycloalkyl group wherein said C₃-C₈ heterocycloalkyl group is optionally substituted with one or more C₁-C₆ alkyl groups, or wherein R₃ is C₃-C₈ heterocycloalkyl;
each R₉ and R₁₀ is independently selected from H or C₁-C₆ alkyl; and
each R₁₁ and R₁₂ is independently selected from H or C₁-C₆ alkyl.

The additive may be a compound of Formula IX

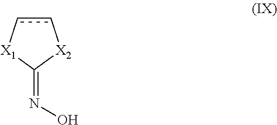

wherein X₁ and X₂ are each independently selected from 0 or NH, preferably wherein at least one of X₁ and X₂ is NH, and
wherein the dashed bond is absent (in which case the compound of Formula IX is a compound of Formula IXa

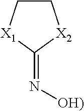

or present (in which case the compound of Formula IX is a compound of Formula IXb

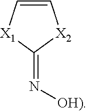

The additive may be a compound of Formula X

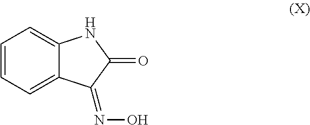 [SMILES: O=C1Nc2ccccc2/C1=N/O]

optionally substituted with one or more substituents selected from C₁-C₆ alkyl or aryl (preferably a 6- to 10-membered aryl, more preferably phenyl). Preferably the substituent is on the nitrogen of the indolinone ring.

The additive may be a compound of Formula XI

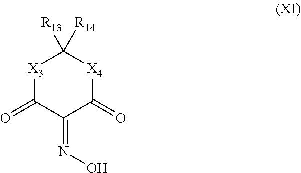

wherein X₃ and X₄ are independently selected from O and NR₁₉,
R₁₉ is H or C₁-C₆ alkyl, preferably C₁-C₆ alkyl, and
R₁₃ and R₁₄ are independently selected from H or C₁-C₆ alkyl, or R₁₄ is absent and R₁₃ is ═O or =S.

The additive may be a compound of Formula XII

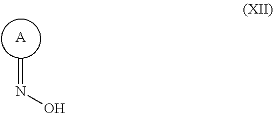

wherein Ring A is a fused tricyclic ring comprising 10-14 ring members which may be selected from carbon ring members and one or more if N, O or S. The nitrogen of the oxime shown in Formula XII is double bonded to any available carbon ring member in Ring A. Ring A may be substituted by one or more substituents selected from C₁-C₆ alkyl, C₁-C₆ haloalkyl and =N—OH. When the substituent is ═N**—OH the nitrogen marked with ** is double bonded to any available carbon ring member of ring A. The one or more substituents are preferably =N—OH. The compound of Formula XII may be a compound of Formula XIa or XIIb

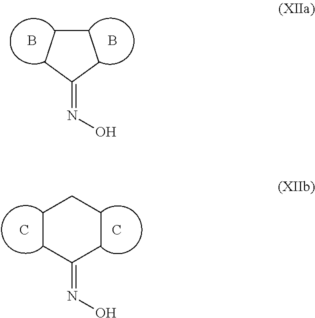

wherein each Ring B and Ring C is independently phenyl or 5-6 membered heteroaryl (preferably pyridyl). Formula XIa and XIIb may be substituted with one or more substituents as set out above for Formula XII.

The additive may be phenol, optionally substituted with one or more halogen atoms (preferably wherein the halogen atoms are each independently selected from F and Cl) or one or more electron withdrawing groups such as nitro. Preferably, the phenol is substituted. Examples of suitable additives having this structure are pentafluorophenol, 2,3,5-trichlorophenol and 4-nitrophenol.

The additive may be a compound of Formula XIII

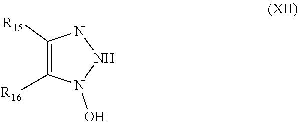

wherein each R₁₅ and R₁₆ is independently selected from H, halogen (preferably Cl), (C₂-C₆ alkylcarbonyl)C₁-C₆ alkyl (i.e. C₃-C₁₂ alkylcarbonylalkyl), —C(O)OC₁-C₆ alkyl, or —C(O)C₁-C₆ alkyl, or wherein R₁₅ and R₁₆ are taken together to form an aryl ring (preferably a 6- to 10-membered aryl, more preferably phenyl) or a 5 or 6-membered heteroaromatic ring comprising one or more heteroatoms selected from O, N or S, optionally wherein the aryl or heteroaromatic ring is substituted with one or more substituents selected from halogen, C₁-C₆ haloalkyl, or nitro. Preferably the ring formed by R₁₅ and R₁₆ together is phenyl or pyridyl. For example, the compound of Formula XIII may be 4-aza-, 5-aza-, 6-aza- or 7-aza-1-hydroxybenzotriazole.

The additive may be a compound of Formula XIV

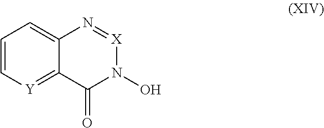

wherein (1) Y is CH and X is N, or (2) Y is N and X is CH, or (3) Y is N and X is N.

The additive may be a compound of Formula XV

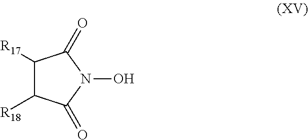

wherein R₁₇ and R₁₈ are H or wherein R₁₇ and R₁₈ are taken together to form a C₅-C₁₀ cycloalkyl or C₅-C₁₀ cycloalkenyl ring or an aryl ring (preferably a 6- to 10-membered aryl, more preferably phenyl).

The additive may be a hydroxypyridine-N-oxide, preferably 2-hydroxypyridine-N-oxide.

The additive may be a hydroxytetrazole, preferably 2H-tetrazol-2-ol.

The additive may be a 1H-benzo[d]imidazol-1-ol optionally substituted with one or more halogen or phenyl groups. Preferably, the phenyl substituent is present and at the 2-position of the imidazole ring. If present, the halogen is preferably Cl.

The additive may be a 1-hydroxyindolin-2-one, optionally substituted with one or more halogen or phenyl groups. If present, the halogen is preferably Cl. The 1-hydroxyindolin-2-one is preferably unsubstituted.

The additive may be a compound selected from those listed in Table 1:

| | | |
|---|---|---|
| 1 | ethyl 2-cyano-2-(hydroxyimino)acetate (OxymaPure/Oxyma) | 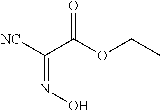 |
| 2 | tert-butyl 2-cyano-2-(hydroxyimino)acetate | 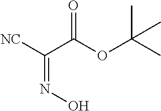 |
| 3 | (2,2-dimethyl-1,3-dioxolan-4-yl)methyl 2-cyano-2- (hydroxyimino)acetate | 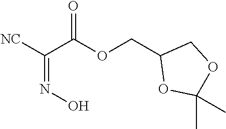 [SMILES: CC1(C)OCC(COC(=O)/C(C#N)=N\O)O1] |
| 4 | 2-amino-N-hydroxy-2-oxoacetimidoyl cyanide | 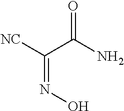 |
| 5 | 2-(ethylamino)-N-hydroxy-2-oxoacetimidoyl cyanide | 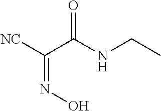 |
| 6 | 2-(dimethylamino)-N-hydroxy-2-oxoacetimidoyl cyanide | 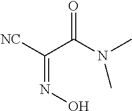 |
| 7 | N-hydroxy-2-(piperidin-1-yl)-2-oxoacetimidoyl cyanide | 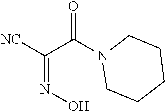 [SMILES: N#C/C(=N/O)C(=O)N1CCCCC1] |
| 8 | N-hydroxy-2-(morpholino)-2-oxoacetimidoyl cyanide | 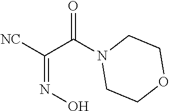 [SMILES: N#C/C(=N/O)C(=O)N1CCOCC1] |
| 9 | 5-(hydroxyimino)-1,3-dimethylpyrimidine- 2,4,6(1H,3H,5H)-trione | 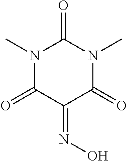 |
| 10 | 1,3-diethyl-5-(hydroxyimino)-2- thioxodihydropyrimidine-4,6(1H,5H)-dione | 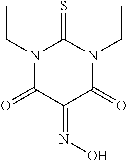 |
| 11 | hydroxyimino-2-phenylacetonitrile | 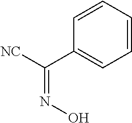 [SMILES: N#C/C(=N/O)c1ccccc1] |
| 12 | hydroxyimino-2-(4-chlorophenyl)acetonitrile | 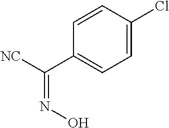 [SMILES: N#C/C(=N/O)c1ccc(Cl)cc1] |
| 13 | N-hydroxypicolinimidoyl cyanide | 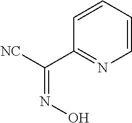 [SMILES: N#C/C(=N/O)c1ccccn1] |
| 14 | hydroxyimino-2-(naphth-1-yl)acetonitrile | 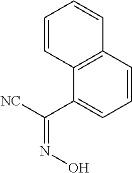 [SMILES: N#C/C(=N/O)c1cccc2ccccc12] |
| 15 | N-hydroxycarbonimidoyl dicyanide | 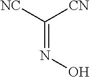 |
| 16 | 1-ethyl 3-methyl-2-(hydroxyimino)malonate | 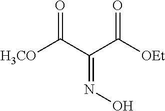 [SMILES: CCOC(=O)/C(=N\O)C(=O)OC] |
| 17 | diethyl(hydroxyimino)malonate | 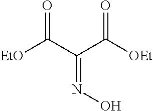 [SMILES: CCOC(=O)C(=NO)C(=O)OCC] |
| 18 | diisopropyl(hydroxyimino)malonate | 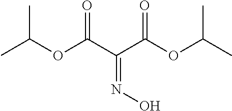 |
| 19 | 5-(hydroxyimino)-2,2-dimethyl-1,3-dioxane-4,6- dione (HONM) | 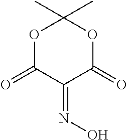 |
| 20 | 1-hydroxypyrrolidine-2,5-dione (HOSu) | 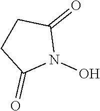 |
| 21 | N-hydroxy-5-norbornene-2,3-dicarboximide (HONB) | 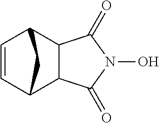 |
| 22 | 2-pyridinol 1-oxide (HOPO which has CAS no 13161-30-3) | 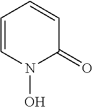 |
| 23 | 2-phenyl-1H-benzo[d]imidazol-1-ol (HOBI) | 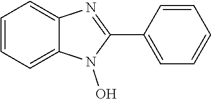 [SMILES: On1c(-c2ccccc2)nc2ccccc21] |
| 24 | 6-chloro-2-phenyl-1H-benzo[d]imidazol-1-ol (6-Cl- HOBI) | 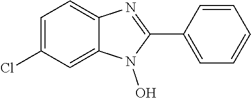 [SMILES: On1c(-c2ccccc2)nc2ccc(Cl)cc21] |
| 25 | 3-hydroxypyrido[3,2-d]pyrimidin-4(3H)-one (HODhad) | 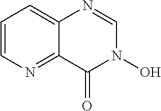 [SMILES: O=c1c2ncccc2ncn1O] |
| 26 | 2H-tetrazol-2-ol | 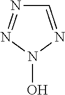 |
| 27 | 1-hydroxyindolin-2-one (HOI) | 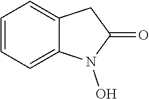 [SMILES: O=C1Cc2ccccc2N1O] |
| 28 | 2-(hydroxyimino)-2-(pyrazin-2-yl)acetamide | 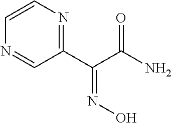 [SMILES: NC(=O)/C(=N\O)c1cnccn1] |
| 29 | ethyl-2-(hydroxyimino)-2-(pyrazin-2-yl)acetate | 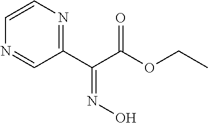 |
| 30 | ethyl-2-(furan-2-yl)-2-(hydroxyimino)acetate | 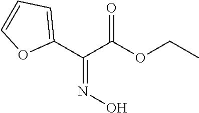 |
| 31 | 2-(furan-2-yl)-2-(hydroxyimino)acetamide | 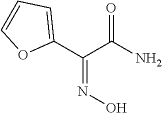 [SMILES: NC(=O)/C(=N\O)c1ccco1] |
| 32 | ethyl 2-(hydroxyimino)-2-(thiazol-2-yl)acetate | 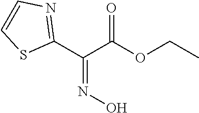 |
| 33 | 2-(hydroxyimino)-2-(thiazol-2-yl)acetamide | 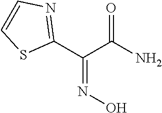 [SMILES: NC(=O)/C(=N\O)c1nccs1] |
| 34 | ethyl 2-(hydroxyimino)-2-(pyrimidin-2-yl)acetate | 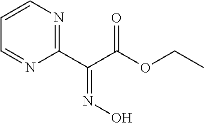 |
| 35 | 2-(hydroxyimino)-2-(pyrimidin-2-yl)acetamide | 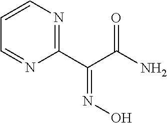 [SMILES: NC(=O)/C(=N\O)c1ncccn1] |
| 36 | 2-(hydroxyimino)-2-(pyrimidin-2-yl)ethanethioamide | 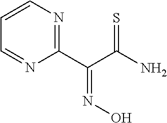 [SMILES: NC(=S)/C(=N\O)c1ncccn1] |
| 38 | 2-(hydroxyimino)-2-(1H-pyrrol-2-yl)acetamide | 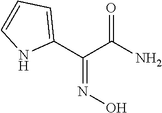 [SMILES: NC(=O)/C(=N\O)c1ccc[nH]1] |
| 39 | 2-(hydroxyimino)-2-(thiophen-2-yl)acetamide | 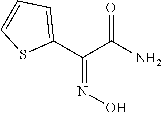 [SMILES: NC(=O)/C(=N\O)c1cccs1] |
| 40 | ethyl 2-(hydroxyimino)-2-(1H-pyrrol-2-yl)acetate | 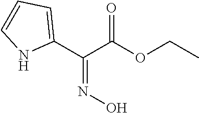 |
| 41 | ethyl 2-(hydroxyimino)-2-(thiophen-2-yl)acetate | 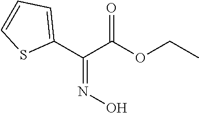 |
| 42 | ethyl 2-(hydroxyimino)-2-(pyridin-2-yl)acetate | 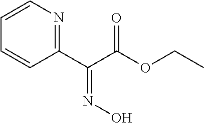 |
| 43 | ethyl 2-(hydroxyimino)-2-(pyridin-4-yl)acetate | 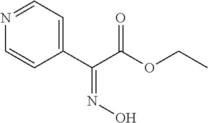 |
| 44 | 2-(hydroxyimino)but-3-ynamide | 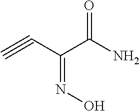 |
| 45 | ethyl 2-(hydroxyimino)but-3-ynoate | 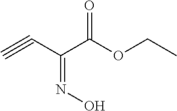 |
| 46 | ethyl 2-(hydroxyimino)-2-nitroacetate | 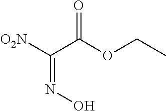 |
| 47 | dinitromethanone oxime | 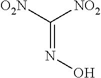 |
| 48 | nitro(phenyl)methanoneoxime | 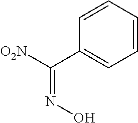 [SMILES: O=[N+]([O-])/C(=N/O)c1ccccc1] |
| 49 | 1-nitro-3-phenylpropan-1-one oxime | 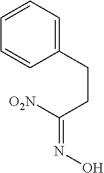 [SMILES: O=[N+]([O-])/C(CCc1ccccc1)=N/O] |
| 50 | 1-(hydroxyimino)-N,N-dimethyl-1- phenylmethanesulfinamide | 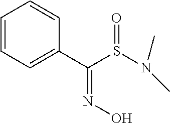 |
| 51 | ethyl 2-(hydroxyimino)-2-sulfamoylacetate | 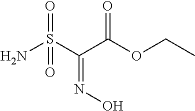 |
| 52 | (hydroxyimino)(methylsulfonyl)methanesulfinamide | 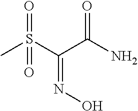 |
| 53 | 2-(hydroxyimino)-2-(methylsulfonyl)acetamide | 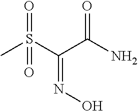 |
| 54 | ethyl 2-(hydroxyimino)-2- ((trifluoromethyl)sulfonyl)acetate | 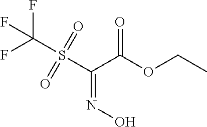 |
| 55 | 2-ethoxy-N-hydroxy-2-oxoacetimidic trifluoromethanesulfonic anhydride | 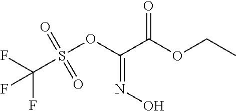 [SMILES: CCOC(=O)/C(=N\O)OS(=O)(=O)C(F)(F)F] |
| 56 | diphenylmethanone oxime | 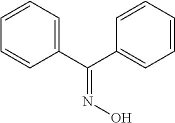 [SMILES: ON=C(c1ccccc1)c1ccccc1] |
| 57 | 2-((hydroxyimino)(pyridin-2-yl)methyl)-1- methylpyridin-1-ium iodide | 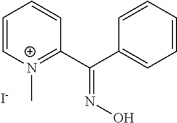 [SMILES: [I-] ; C[n+]1ccccc1/C(=N/O)c1ccccc1] |
| 58 | 9H-fluoren-9-one oxime | 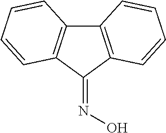 [SMILES: ON=C1c2ccccc2-c2ccccc21] |
| 59 | anthracene-9,10-dione dioxime | 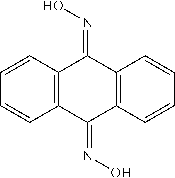 [SMILES: ON=C1c2ccccc2C(=NO)c2ccccc21] |
| 60 | pyrido[3,4-g]isoquinoline-5,10-dione dioxime | 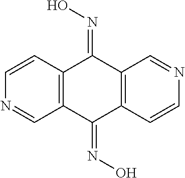 [SMILES: O/N=C1\c2ccncc2/C(=N/O)c2ccncc21] |
| 61 | imidazolidin-2-one oxime | 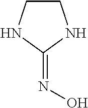 |
| 62 | 1,3-dihydro-2H-imidazol-2-one oxime | 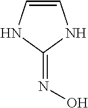 |
| 63 | oxazolidin-2-one oxime | 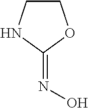 |
| 64 | oxazol-2(3H)-one oxime | 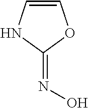 |
| 65 | 2-(hydroxyimino)malonamide | 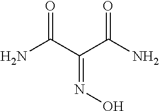 |
| 66 | N¹,N³-dihydroxy-2-(hydroxyimino)malonamide | 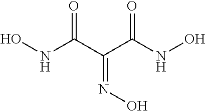 [SMILES: O=C(NO)C(=NO)C(=O)NO] |
| 67 | butane-2,3-dione dioxime | 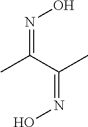 |
| 68 | N¹,N²-dihydroxyoxalimidoyl dichloride | 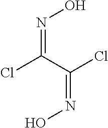 |
| 69 | N′-hydroxypyrazine-2-carboximidamide | 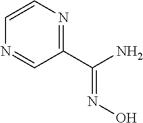 |
| 70 | 3-methyl-1,2,4-oxadiazole-5-carbaldehyde oxime | 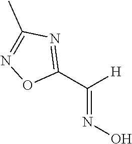 |
| 71 | 3-(hydroxyimino)-1-phenylindolin-2-one | 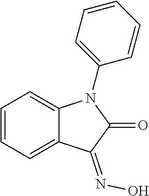 [SMILES: O=C1/C(=N\O)c2ccccc2N1c1ccccc1] |
| 72 | 3-(hydroxyimino)-1-methylindolin-2-one | 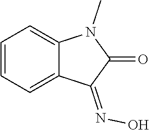 |
| 73 | 2-((hydroxyimino)methyl)-1-methylpyridin-1-ium | 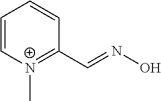 |
| 74 | 2,3,4,5,6-pentafluorophenol | 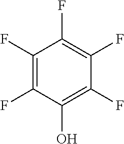 [SMILES: Oc1c(F)c(F)c(F)c(F)c1F] |
| 75 | 2,3,5-trichlorophenol | 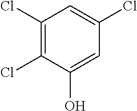 |
| 76 | 4-nitrophenol | 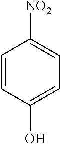 |
| 77 | 1-hydroxybenzotriazole (HOBt) | 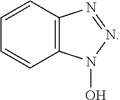 |
| 78 | 1-hydroxy-6-nitrobenzotriazole (6-NO₂-HOBt) | 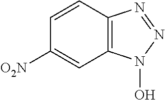 [SMILES: O=[N+]([O-])c1ccc2nnn(O)c2c1] |
| 79 | 6-trifluoromethyl-1-hydroxy-benzotriazole (6-CF₃- HOBt) | 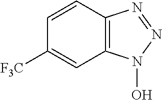 [SMILES: On1nnc2ccc(C(F)(F)F)cc21] |
| 80 | 1-hydroxy-7-azabenzotriazole (HOAt) | 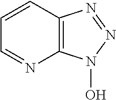 |
| 81 | 6-chloro-1-hydroxy-benzotriazole (6-Cl-HOBt) | 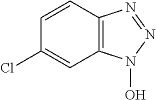 [SMILES: On1nnc2ccc(Cl)cc21] |
| 82 | 5-aza-1-hydroxybenzotriazole (6-HOAt) | 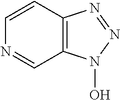 |
| 83 | 6-aza-1-hydroxybenzotriazole (5-HOAt) | 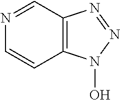 |
| 84 | 4-aza-1-hydroxybenzotriazole (4-HOAt) | 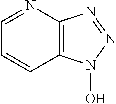 |
| 85 | 3,4-dihydro-3-hydroxy-4-oxo-1,2,3-benzotriazine (HODhbt) | 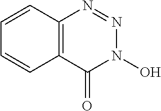 [SMILES: O=c1c2ccccc2nnn1O] |
| 86 | 3-hydroxy-4-oxo-3,4-dihydro-5-azabenzo-1,2,3- triazine (HODhat) | 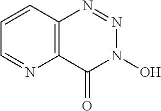 [SMILES: O=c1c2ncccc2nnn1O] |
| 87 | 1-hydroxy-1H-1,2,3-triazole | 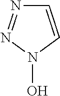 |
| 88 | 5-chloro-1-hydroxy-1H-1,2,3-triazole | 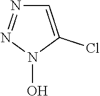 |
| 89 | 5-acetyl-1-hydroxy-1H-1,2,3-triazole | 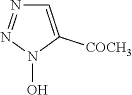 |
| 90 | 1-(1-hydroxy-1H-1,2,3-triazol-5-yl)propan-2-one | 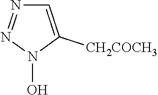 |
| 91 | ethyl-1-hydroxy-1H-1,2,3-triazole-4-carboxylate | 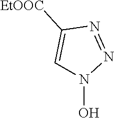 [SMILES: CCOC(=O)c1cn(O)nn1] |

In some embodiments, the additive is not HOAt.

Preferably, the additive is selected from the group consisting of OxymaPure, HOBt, HOSu, HOPO, pentafluorophenol, and 6-CI-HOBt, more preferably OxymaPure, HOBt, HOSu and HOPO, more preferably HOPO.

In some embodiments, the carbodiimide-additive combination is not DIC-HOPO.

As set out above, the additive reacts with the O-acylisourea intermediate to provide an activated ester. When the additive is a compound of Formula (IV) the activated ester is a compound of Formula (V) or a salt thereof:

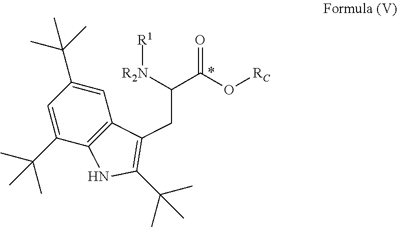

R_{C} in Formula (V) may correspond to the non-hydroxyl portion of the additives listed in Table 1.

### Reaction Conditions

The reaction between the compound of Formula (I) or the salt thereof and the carbodiimide reagent, and/or the reaction between the O-acylisourea intermediate and the additive, and/or the reaction between the activated ester and the amino-containing moiety, may be carried out at a temperature of from −10 to 40° C., preferably from 0 to 30° C., such as from 0 to 25° C. Typically, these reactions are carried out in a one-pot procedure/successively in the same reaction vessel. In other words, all reagents for all the steps of the method recited in claim 1 are typically added to form a single reaction mixture (one-pot). Thus, preferably each of the above reactions are carried out at a temperature of from −10 to 40° C., preferably from 0 to 30° C., such as from 0 to 25° C. The reactions are typically carried out at around 1 atm of pressure. In some embodiments, the reagents are mixed at around 2.5° C. and then heated to room temperature. Thus, the temperatures can vary within the above ranges during the reactions.

The inventors surprisingly found that these relatively low temperatures can be used to couple an amino-containing moiety as defined herein to a compound of Formula (I) in high yields, despite the extremely sterically bulky side chain of the Tbt residue in Formula (I). In the processes described herein, it is desirable to avoid elevated temperatures to reduce the risk of side reactions which lead to epimerisation.

The reactions between the compound of Formula (I) or the salt thereof and the carbodiimide reagent, between the O-acylisourea intermediate and the additive, and between the activated ester and the amino-containing moiety may be conducted within a total duration of less than 48 hours, preferably as less than 36 hours, more preferably less than 24 hours. In some embodiments, the total duration of the above reactions is 2-48 hours, preferably 4-36 hours, more preferably 10-24 hours, such as about 18 or about 20 hours.

As the reactions between the compound of Formula (I) or the salt thereof and the carbodiimide reagent, between the O-acylisourea intermediate and the additive, and between the activated ester and the amino-containing moiety are typically carried out in a one-pot procedure, the O-acylisourea and activated ester intermediates are preferably not isolated.

The carbodiimide reagent may be added last, after the compound of Formula (I) or the salt thereof, the amino-containing moiety, the additive, solvent(s), and any optional acid or base have been mixed. The reaction mixture may be cooled before the carbodiimide reagent is added.

In some embodiments, the carbodiimide reagent is added last to initiate the reactions.

### Acidic Conditions

The reactions between the compound of Formula (I) or the salt thereof and the carbodiimide reagent, between the O-acylisourea intermediate and the additive, and between the activated ester and the amino-containing moiety may be carried out in the presence of an acid, or a base, or no acid or base may be added. If used, suitable bases include: DIPEA, N-methylmorpholine, pyridine, trimethylamine and 2,4,6-trimethylpyridine.

Preferably, the reactions between the compound of Formula (I) or the salt thereof and the carbodiimide reagent, between the O-acylisourea intermediate and the additive, and between the activated ester and the amino-containing moiety are carried out under acidic conditions. The inventors have surprisingly found that addition of acid improves conversion of the compound of Formula (I) (e.g. overnight or within a total duration as defined above of 4-36 hours, preferably within 10-24 hours). This was contrary to the inventor's initial expectation that adding acid would hinder the process due to protonation of the amino group of the amino-containing moiety.

Preferably, the reaction between the compound of Formula (I) or the salt thereof and the carbodiimide reagent, and/or the reaction between the O-acylisourea intermediate and/or the reaction between the additive, and/or between the activated ester and the amino-containing moiety, is acid-catalysed. Acid catalysts may enhance the electrophilicity of the carbonyl marked * in Formula (I) and (V) (and the equivalent carbonyl in Formula (III)) and thereby increase the rate of nucleophilic acyl substitution reactions at this carbonyl, for example the reaction forming the O-acylisourea.

Without wishing to be bound by theory, the inventors postulate that increasing the rate of the reaction of the compound of Formula (I) or the salt thereof and the carbodiimide reagent to form the O-acylisourea may compensate for any protonation of the amino group of the amino-containing moiety. Thus, the reaction between the compound of Formula (I) or the salt thereof and the carbodiimide reagent is preferably acid-catalysed.

Preferably, the reactions between the compound of Formula (I) or the salt thereof and the carbodiimide reagent, the O-acylisourea intermediate and the additive, and between the activated ester and the amino-containing moiety are carried out at a pH of less than 6.5, preferably 1-6.5, more preferably 1.5-6, more preferably 4-6. These reactions may also be carried out at a pH of less than 5, such as 1-4.5, 1.5-4, or 2-3.

When the above reactions are carried out in non-aqueous solvent, the pH is determined by taking a sample from the reaction mixture after the addition of the acid (preferably immediately after addition of the acid), adding water (preferably wherein ratio of the added water to the sample of the reaction mixture is from 1:10 to 10:1 v/v, preferably 1:1 to 10:1 v/v, more preferably about 10:1 v/v) and mixing, optionally separating the aqueous phase, and measuring the pH of the aqueous phase or of the reaction mixture containing water. Optionally, a sample may also be taken from the reaction mixture before addition of the acid for use as an in-process control. When the reactions are carried out in non-aqueous solvent, pH may be determined by Test Method B as described in ASTM D4980.

Preferably, the reactions between the compound of Formula (I) or the salt thereof and the carbodiimide reagent, the O-acylisourea intermediate and the additive, and between the activated ester and the amino-containing moiety are carried out in the presence of at least 0.1 equivalents of acid, more preferably at least 0.25 equivalents of acid, more preferably at least 0.5 equivalents of acid per equivalent of Formula (I) or the salt thereof, more preferably at least 0.8 equivalents of acid per equivalent of Formula (I) or the salt thereof. For example, the reactions may be carried out in the presence of 0.25-2, preferably 0.5-1.5, more preferably 0.8-1.2 equivalents of acid per equivalent of Formula (I) or the salt thereof. In other words, at least 0.1 equivalents of acid, preferably at least 0.25 equivalents of acid, more preferably at least 0.5 equivalents of acid, more preferably at least 0.8 equivalents of acid may be added per equivalent of Formula (I) or the salt thereof. For example, 0.25-2, preferably 0.5-1.5, more preferably 0.8-1.2 equivalents of acid may be added per equivalent of Formula (I) or the salt thereof. Optionally, in these embodiments, the acid may be any of the acids listed below, preferably HCl (dioxane), such as 4N HCl (dioxane).

The acid may be a strong acid having a pKa of less than 1. Examples of suitable acids include hydroiodic acid, hydrobromic acid, perchloric acid (HClO₄), hydrochloric acid, chloric acid (HClO₃), sulfuric acid and nitric acid. Preferably, the acid is hydrochloric acid, more preferably anhydrous hydrochloric acid such as HCl (dioxane).

The reactions between the compound of Formula (I) or the salt thereof and the carbodiimide reagent, the O-acylisourea intermediate and the additive, and the activated ester and the amino-containing moiety may be carried out in any suitable solvent(s). Suitable solvents include aqueous or non-aqueous solvents, non-aqueous solvents are preferred. Examples of suitable solvents include water, dichloromethane (DCM), dimethylformamide (DMF), N,N-dimethylacetamide (DMA), acetonitrile (ACN), N-methylpyrrolidone, dimethylsulfoxide, 2-methyltetrahydrofuran (MeTHF), dioxane or a mixture thereof, such as a mixture of DMA, dioxane, and optionally water. The solvent may preferably comprise one or more polar aprotic solvents (such as one or more of: DCM, DMF, DMA, ACN, methylpyrrolidone, dimethylsulfoxide, and MeTHF), more preferably, the solvent comprises DMA.

The amino-containing moiety is preferably added in the form of a solution. In some preferred cases, the amino-containing moiety may be added in the form of a solution comprising DMA solvent. Preferably, the solution containing the amino-containing moiety comprises less than 10 wt % of water, more preferably less than 7.5 wt % of water, more preferably less than 5 wt % of water. In some embodiments, no water solvent is added in the process (i.e. when performing the reactions recited in claim 1) other than any water present in the solution comprising the amino-containing moiety and any water entrained in the compound or Formula (I) or the salt thereof which is typically added as a solid.

Preferably, the solvent is substantially free of water, e.g. the solvent in the reaction mixture, and thus the reaction mixture overall, may comprise less than 10 wt % of water, such as less than 5 wt % of water, such as less than 3 wt % or less than 1 wt % of water. As illustrated above, relatively low amounts of water may be present in the reagents when added but preferably no additional water solvent is added to the reaction mixture.

In other preferred embodiments, the solvent is substantially free of water, methanol and ethanol, e.g. the solvent in the reaction mixture, and thus the reaction mixture overall, may comprise water, methanol and ethanol in a total amount of less than 10 wt %, such as less than 5 wt %, such as less than 3 wt % or less than 1 wt %.

Preferably, the solvent in the reaction mixture, and thus the reaction mixture overall, comprises at least 70 wt %, more preferably at least 80 wt %, more preferably at least 90 wt % of one or more polar aprotic solvents (such as one or more of: DCM, DMF, DMA, ACN, methylpyrrolidone, dimethylsulfoxide, and MeTHF). In this embodiment, the polar aprotic solvent is preferably DMA.

The method of the invention may further comprise one or more purification steps.

The method of the invention may further comprise preparing the compound of Formula (I). For example, when R¹ is a peptide or an amino acid, the method may further comprise coupling said peptide or amino acid to the Tbt residue. Any suitable peptide coupling technique may be used to link the R¹ group to the Tbt residue. In some cases, the compound of Formula (I) may be prepared by pre-activating the R¹ amino acid or peptide, such as with pivaloyl chloride or isobutylchloroformate, and then coupling with Tbt, optionally silylated Tbt. Peptide production methods involving silylated peptides are disclosed in WO2009/065836.

The method of the invention may also further comprise preparing the amino-containing moiety. For example, when the amino-containing moiety comprises the C-terminal capping group, such as a capping group having formula —X—Y—Z as defined above (e.g. —NHCH₂CH₂Ph), the process may comprise activating the C-terminal carboxylic acid group of the amino-containing moiety, which typically comprises an amino protecting group such as Cbz, and then coupling to H—X—Y—Z (e.g. H₂NCH₂CH₂Ph). Suitable activators for this step include pivaloyl chloride or isobutylchloroformate.

### Target Peptide

A target peptide according to the present invention will typically have a chain length of up to 20 amino acids. Preferably, target peptides are 2 to 10, 3 to 7 or 3 to 5, e.g. 3 amino acids in length.

The target peptide is preferably an antimicrobial peptide.

Preferably, the target peptide is a compound of Formula (VII) or a salt thereof

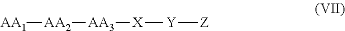

wherein:
each AA₁ and AA₃ is independently a cationic amino acid, preferably lysine or arginine but may be histidine or any non-genetically coded or modified amino acid carrying a positive charge at pH 7.0;
AA₂ is Tbt, i.e.

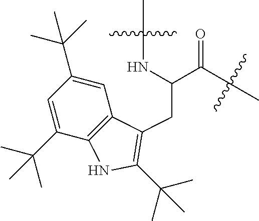 [SMILES: *NC(Cc1c(C(C)(C)C)[nH]c2c(C(C)(C)C)cc(C(C)(C)C)cc12)C(*)=O]

wherein the left hand wiggly bond denotes the point of attachment to AA₁ and the right hand wiggly bond denotes the point of attachment to AA₃-X—Y—Z; and
X, Y and Z are as defined above.

Non-genetically coded or modified amino acids that are suitable as AA₁ and/or AA₃ are set out above.

The compounds of Formula (VII) are antimicrobial peptides and are disclosed in WO2009/081152.

The target peptide may include all enantiomeric forms, both D and L amino acids and enantiomers resulting from chiral centres within the amino acid R groups and moieties Y or Z, when present.

Preferably, the target peptide is Arg-Tbt-Arg-NHCH₂CH₂Ph, i.e. the compound

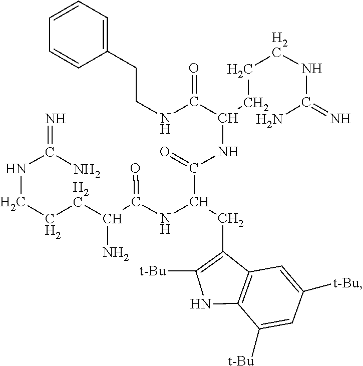 [SMILES: CC(C)(C)c1cc(C(C)(C)C)c2[nH]c(C(C)(C)C)c(CC(NC(=O)C(N)CCCNC(=N)N)C(=O)NC(CCCNC(=N)N)C(=O)NCCc3ccccc3)c2c1]

or a salt thereof. Most preferably, the target peptide is a compound having the following structure:

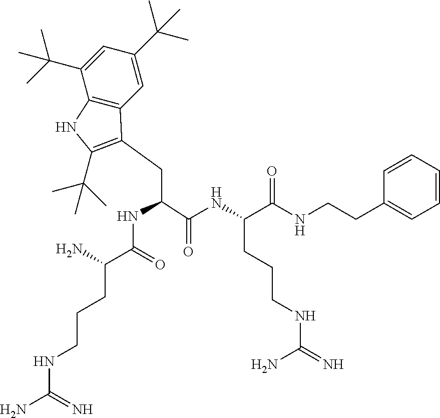 [SMILES: CC(C)(C)c1cc(C(C)(C)C)c2[nH]c(C(C)(C)C)c(C[C@H](NC(=O)[C@@H](N)CCCNC(=N)N)C(=O)N[C@@H](CCCNC(=N)N)C(=O)NCCc3ccccc3)c2c1]

(which is also referred to herein as AMC-109) or a salt thereof.

As illustrated in the following Examples, protecting groups, particularly amino protecting groups, may be employed in the methods of the invention. For example, it may be necessary to remove one or more protecting groups from the product of the reaction between the amino-containing moiety and the activated ester to provide the target peptide. The methods of the invention may comprise steps of removing any protecting groups. For example, Cbz protecting groups may be removed by hydrogenolysis with H₂ over palladium on carbon (Pd/C).

### EXAMPLES

### 1.1 Preparation of Intermediates Z-Arq-Tbt-OH (AMC-01) and H-Arq-NHEtPh (AMC-03)

Z-Arg-Tbt-OH (AMC-01) was prepared by activation of Cbz-protected arginine with isobutylchloroformate (IBCF) followed by coupling with silylated Tbt as illustrated in the following scheme.

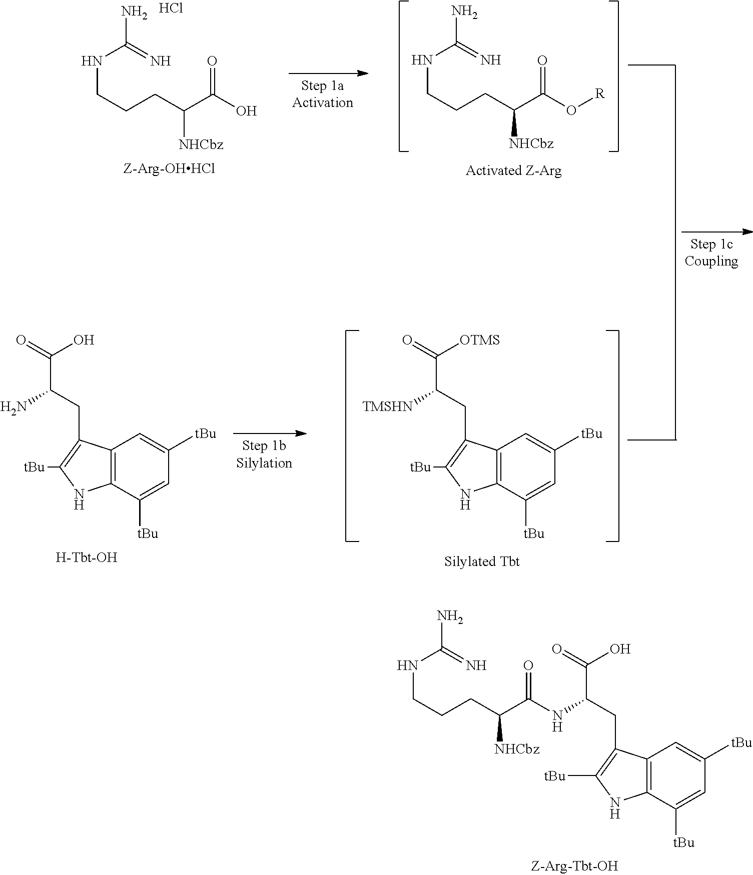

As illustrated in the following scheme, Z-Arg-NHEtPh (AMC-02) was prepared by activating commercially available Z-Arg-OH·HCl with IBCF and reacting activated Cbz-protected arginine with H₂NEtPh, affording AMC-02 in the form of a HCl salt as a 15 white solid following work-up.

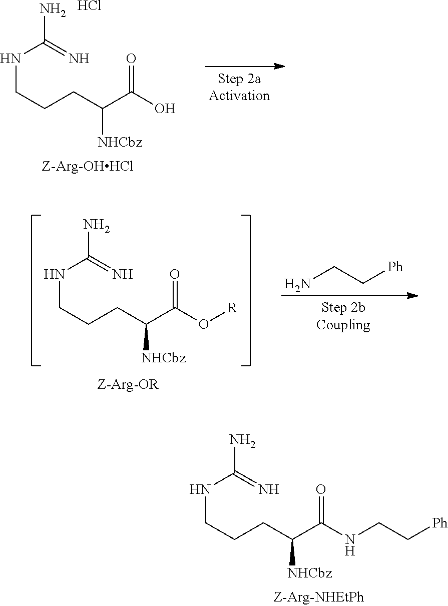

The AMC-02 in the form of the HCl salt was then deprotected to provide H-AraNHEtPh (AMC-03) as illustrated in the followina scheme:

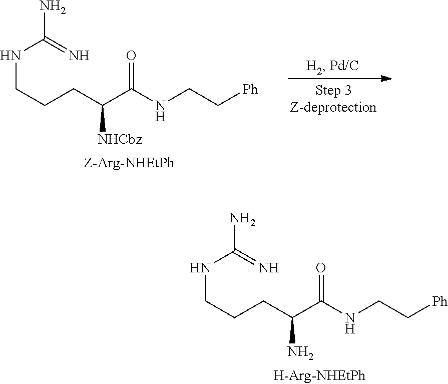 [SMILES: N=C(N)NCCC[C@H](NC(=O)OCc1ccccc1)C(=O)NCCc1ccccc1 ; N=C(N)NCCC[C@H](N)C(=O)NCCc1ccccc1]

The procedure for the deprotection step (Step 3) was as follows. AMC-02 in the form of the HCl salt (37.20 g, 75% wt.), MeOH (550 mL) and water (130 mL) were introduced into a 2 L three neck flask. The suspension was stirred under nitrogen until all of the AMC-02 was fully soluble. Pd/C (10% wt, 50% wet, 2.27 g, 1.5 mol %) was added and the N₂ atmosphere was replaced with H₂ (using a H₂ generator, 0.7 bar). Deprotection conversion was followed by HPLC and full conversion was obtained after 3 h. Pd/C was then filtered, washed twice with MeOH/water (8/2, v/v, 2×75 mL). The filtrates were combined and concentrated under reduced pressure (Tbath=55° C., from 300 mbar to 50 mbar) up to 31 g of concentrated solution. DMA (100 mL) was added and evaporation was pursued (Tbath=65° C., 25 mbar) to afford AMC-03 in the form of a HCl salt as a colourless solution (117 mL, 115 g, 17% wt by NMR, est. 19.6 g net peptide, >99% yield).

### 1.2 Couplinq of Z-Arq-Tbt-OH (AMC-01) and H-Arq-NHEtPh (AMC-03) to Provide Z-Arq-Tbt-Arq-NHEtPh (AMC-04)

In a 250 mL round bottom flask, AMC-01 (20.02 g, 80% wt) was introduced followed by HOPO (2.82 g), DMA (67 mL), AMC-03 in the colourless solution described above (44.10 g, 17% wt) and HCl 4 N in dioxane (6.0 mL). The resulting solution was cooled down to 2.5±2.5° C. and EDC.HCl (5.78 g) was added. The reaction mixture was warmed up to room temperature (RT) and stirred for 20 h. Coupling conversion was followed by HPLC.

The reaction mixture (138 mL) was diluted with water (276 mL) and EtOAc 25 (276 mL). Phases were separated and resulting peptide aqueous phase (460 mL) was diluted with 6N aq. HCl (31 mL, diluted from 12N HCl). Prior to dilution with the 6N aq. HCl, the peptide aqueous phase had a pH of 5.4. NaCl (11.4 g) was added to minimize peptide loss in aqueous phase followed by EtOAc (276 mL). Phases were separated and resulting peptide organic phase (330 mL) was washed twice with aq. 2.5% NaCl (138 mL and 69 mL) and concentrated in a rotary evaporator (Tbath=55° C., 250 mbar, targeted solution weight: 50 g) to give AMC-04 in solution (44.21 g). The pH of the solution containing the AMC-04 was measured as 3.2. The amount of AMC-04 in the solution was determined by 1H NMR. Purity was determined by HPLC.

| Test | Results |
|---|---|
| Quantity (net peptide) | Est. 19.0 g |
| Purity by HPLC | 95.6% (area/area) |
| Assay by 1H NMR | 43% wt |
| Yield | 85% |

The parameters for the HPLC method are set out below:

| Column | Waters XSelect CSH C18 XP, 130 A, 2.5 pm, | | | | | |
|---|---|---|---|---|---|---|
| | 3 × 75 mm | | | | | |
| Oven | 40° C. | | | | | |
| Temperature | | | | | | |
| Wavelength | 220 nm | | | | | |
| Mobile Phases | A: 0.1% TFA in water | | | | | |
| | B: 0.1% TFA in ACN | | | | | |
| Flow Rate | 1 mL/min | | | | | |
| | | Time (min) | | | | |
| | | 0 | 7.5 | 8.1 | 8.2 | 10.7 |
| Gradient | % A | 98 | 0 | 0 | 98 | 98 |
| | % B | 2 | 100 | 100 | 2 | 2 |

Surprisingly, despite the extremely high steric bulk of Tbt, the carbodiimide 10 and additive reagents allowed coupling of Z-Arg-Tbt-OH (AMC-01) and H-Arg-NHEtPh (AMC-03) to provide Z Arg-Tbt-Arg-NHEtPh (AMC-04) in high yield under mild reaction conditions (20 h, 2.5° C. to RT).

1.3 Effect of Acid/Base during Coupling of Z-Arq-Tbt-OH (AMC-01) and H-Arq-NHEtPh (AMC-03) to Provide Z-Ara-Tbt-Ara-NHEtPh (AMC-04)

The effect of conducting the reaction without any acid or base (Tests 1-5), in the presence of base (DIPEA—Tests 6-7), or in the presence of acid (H2I 4N (dioxane)—Tests 8-9) was investigated. The number of equivalents of reagents is quoted vs the number of equivalents of AMC-01 in the table below.

The reactions were conducted using an analogous procedure to Example 1.2, except that for Tests 1-5 no HCl in dioxane was added and for Test 6 and 7 DI PEA base was added instead of the HCl in dioxane. Conversion was tracked by HPLC, The abbreviation o.n. stands for overnight and 2 d stands for 2 days.

| | | | EDC · | Acid/ | Conver- | Conver- |
|---|---|---|---|---|---|---|
| | AMC-03 | HOPO | HCl | base | sion | sion |
| Test # | (eq) | (eq) | (eq) | (eq) | o.n. (%) | 2 d (%) |
| 1 | 1.2 | 1.25 | 1.25 | N/A | 94.0 | 99.3 |
| 2 | 1.15 | 1.25 | 1.25 | N/A | 94.4 | 99.4 |
| 3 | 1.10 | 1.25 | 1.25 | N/A | 95.0 | 99.4 |
| 4 | 1.05 | 1.25 | 1.25 | N/A | 94.9 | 98.7 |
| 5 | 1.2 | 1.05 | 1.25 | N/A | 94.2 | 99.5 |
| 6 | 1.2 | 1.25 | 1.25 | DIPEA | 92.1 | N/A |
| | | | | (0.2) | | |
| 7 | 1.2 | 1.25 | 1.25 | DIPEA | 86.7 | N/A |
| | | | | (0.5) | | |
| 8 | 1.2 | 1.05 | 1.25 | HCl 4N | 99.0 | N/A |
| | | | | (dioxane) | | |
| | | | | (0.5) | | |
| 9 | 1.12 | 1.05 | 1.25 | HCl 4N | 100 | N/A |
| | | | | (dioxane) | | |
| | | | | (1.0) | | |

The inventors initially expected the base to make the nucleophile (AMC-03) more reactive and thereby increase conversion over a shorter reaction time. Similarly, it was expected that adding acid would hinder the reaction due to protonation of the amino group of AMC-03. Surprisingly, addition of acid allowed for much a faster reaction (~95% conversion after 4 h [result not shown] and full conversion after overnight—see Tests 8-9) whilst the addition of base was detrimental for coupling conversion (Tests 6-7).

1. A method of peptide synthesis comprising:
reacting a compound of Formula (I) or a salt thereof with a carbodiimide reagent to form an O-acylisourea intermediate;
reacting the O-acylisourea intermediate with an additive to form an activated ester; and
reacting the activated ester with an amino-containing moiety which is an amino acid, a peptide or a salt thereof comprising an amino group, wherein the amino group forms an amide bond with the carbonyl marked * in Formula (I);
wherein the compound of Formula (I) has the structure: 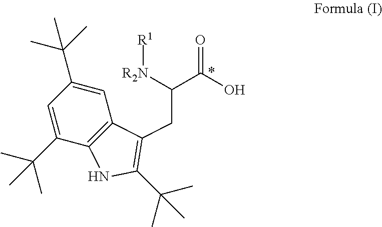
wherein R₁ is a protecting group, a peptide or an amino acid; and
wherein R₂ is H, an alkylsilyl group or a protecting group.

2. The method of claim 1, wherein the reactions between the compound of Formula (I) or the salt thereof and the carbodiimide reagent, the O-acylisourea intermediate and the additive, and the activated ester and the amino-containing moiety are carried out at a temperature of from −10 to 40° C.

3. The method of claim 1, wherein the reactions between the compound of Formula (I) or the salt thereof and the carbodiimide reagent, between the O-acylisourea intermediate and the additive, and between the activated ester and the amino-containing moiety are carried out under acidic conditions.

4. The method of claim 1, wherein the reaction between the compound of Formula (I) or the salt thereof and the carbodiimide reagent, and/or the reaction between the O-acylisourea intermediate and the additive, and/or between the activated ester and the amino-containing moiety, is acid-catalysed.

5. (canceled)

6. The method of claim 1, wherein the reactions between the compound of Formula (I) or the salt thereof and the carbodiimide reagent, the O-acylisourea intermediate and the additive, and between the activated ester and the amino-containing moiety are carried out at a pH of less than 6.5.

7. The method of claim 1, wherein, the reactions between the compound of Formula (I) or the salt thereof and the carbodiimide reagent, the O-acylisourea intermediate and the additive, and between the activated ester and the amino-containing moiety are carried out in the presence of at least 0.1 equivalents of acid per equivalent of the compound of Formula (I) or the salt thereof.

8. The method of claim 1, wherein the amino-containing moiety is provided in the form of a salt.

9. The method of claim 1, wherein the carbodiimide reagent is a compound of Formula (II) or a salt thereof: 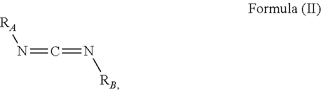
wherein R_{A} and R_{B} are each independently selected from an organic group containing 1-30 non-hydrogen atoms.

10. The method of claim 9, wherein R_{A} and R_{B} are each independently selected from the group consisting of: C₁-C₁₀ alkyl optionally substituted with a mono- or di-(C₁-C₁₀ alkyl)amino group; C₃-C₈ cycloalkyl; aryl; (aryl)C₁-C₁₀alkyl; and (C₃-C₈ heterocycloalkyl)C₁-C₁₀alkyl wherein the heterocycloalkyl group is optionally substituted with one or more C₁-C₁₀ alkyl groups.

11. The method of claim 1, wherein the carbodiimide reagent is 1-ethyl-3-(3-dimethylaminopropyl)carbodiimide hydrochloride (EDC.HCl) or 1-ethyl-3-(3-dimethylaminopropyl)carbodiimide.

12. The method of claim 1, wherein the additive is a compound of Formula IV or a salt thereof: 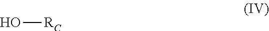
wherein R_{C} is an organic group comprising 1-30 non-hydrogen atoms.

13. The method of claim 12, wherein:
(A) the compound of Formula (IV) is an N-hydroxy compound wherein R_{C} is an organic group containing 1-30 non-hydrogen atoms and at least one nitrogen atom and wherein the hydroxyl group in Formula (IV) is bonded to the R_{C} group through said nitrogen atom, optionally wherein the N-hydroxy compound comprises: a hydroxyimino group, an N-hydroxy-triazole group, an N-hydroxy-tetrazole group, an N-hydroxybenzimidazole group, an N-hydroxyindolin-2-one group, a N-hydroxypyridinone group, an N-hydroxypyrrolidine-2,5-dione group, a 4-aza 5-aza 6-aza or 7-aza-1-hydroxybenzotriazole group, a N-hydroxybenzotriazole group or the group 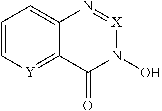
wherein Y is CH and X is N, or Y is N and X is CH, or Y is N and X is N; or
(B) R_{C} is a phenyl group, optionally substituted with one or more halogen atoms or one or more electron withdrawing groups.

14. The method of claim 1, wherein the additive is selected from the compounds in Table 1.

15. The method of claim 1, wherein the additive is 2-pyridinol 1-oxide (HOPO).

16. (canceled)

17. The method of claim 1, wherein R₁ is a cationic amino acid AA₁ optionally comprising one or more protecting groups.

18. The method of claim 1 wherein the amino-containing moiety comprises one or more protecting groups and/or a C-terminal capping group.

19. The method of claim 1, wherein the amino-containing moiety is a compound of Formula (IV) or a salt thereof 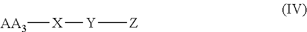
wherein:
AA₃ is a cationic amino acid;
X is a N atom, which may be substituted by a branched or unbranched C₁-C₁₀ alkyl or aryl group, and this alkyl or aryl group may incorporate up to 2 heteroatoms selected from N, O and S;
Y represents a group selected from —Rₐ—R_{b}—, —Rₐ—R_{b}—R_{b}— and —R_{b}—R_{b}—Rₐ— wherein
Rₐ is C, O, S or N, and
R_{b} is C; each of Rₐ and R_{b} may be substituted by C₁-C₄ alkyl groups or unsubstituted; and
Z is a group comprising 1 to 3 cyclic groups each of 5 or 6 non-hydrogen atoms, 2 or more of the cyclic groups may be fused and one or more of the cyclic groups may be substituted;
the Z moiety incorporates a maximum of 15 non-hydrogen atoms; and wherein the bond between Y and Z is a covalent bond between Rₐ or R_{b} of Y and a non-hydrogen atom of one of the cyclic groups of Z.

20. (canceled)

21. (canceled)

22. The method of claim 1, wherein the reactions between the compound of Formula (I) or the salt thereof and the carbodiimide reagent, the O-acylisourea intermediate and the additive, and between the activated ester and the amino-containing moiety are carried out in a solvent which comprises water, methanol and ethanol in a total amount of less than 10 wt %.

23. A method of making a target peptide comprising the method of peptide synthesis of claim 1, wherein the target peptide is 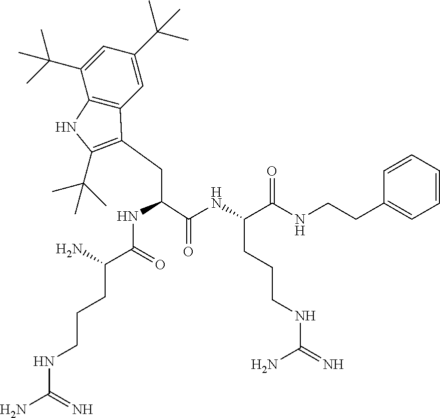 [SMILES: CC(C)(C)c1cc(C(C)(C)C)c2[nH]c(C(C)(C)C)c(C[C@H](NC(=O)[C@@H](N)CCCNC(=N)N)C(=O)N[C@@H](CCCNC(=N)N)C(=O)NCCc3ccccc3)c2c1]
or a salt thereof.

24. A compound of Formula (III) or a salt thereof: 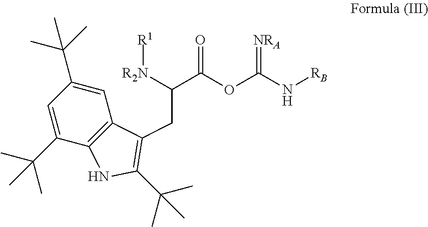
wherein R₁ is a protecting group, a peptide or an amino acid;
wherein R₂ is H, an alkylsilyl group or a protecting group; and
wherein R_{A} and R_{B} are each independently selected from an organic group containing 1-30 non-hydrogen atoms.
